(19) United States
(12) Patent Application Publication
(10) Pub. No.: US 2025/0041121 A1
(43) Pub. Date: Feb. 6, 2025

(54) VOICE ACTIVATED PROTECTIVE HELMET

(71) Applicant: Newtronix LLC, Reseda, CA (US)

(72) Inventor: Jeffrey T. Newton, Reseda, CA (US)

(73) Assignee: Newtronix LLC, Reseda, CA (US)

(21) Appl. No.: 18/229,613

(22) Filed: Aug. 2, 2023

(51) Int. Cl. A61F 9/04; A61F 9/06

(52) U.S. Cl. CPC A61F 9/06

(57) ABSTRACT

A voice actuated helmet is described having a protective shell and a frontal window. A reciprocating shield pivots in front of and away from the frontal window using a motor inside the helmet. The motor is controlled by a processor with a voice recognition module that receives audio signals from a microphone inside the helmet, and converts the voice commands to a signal recognized by the motor to move the shield up and down.

### BACKGROUND

A welding helmet is a kind of personal protective headgear worn when using an acetylene torch to protect the eyes, face, and neck from flash burn, sparks, infrared and ultraviolet light, and intense heat. Welding helmets have been around in the U.S. since the 1930s and are preferably lightweight while thermally insulating to protect against heat and flying embers. These headgear are most commonly used in arc welding processes such as shielded metal arc welding, gas tungsten arc welding, and gas metal arc welding. They are necessary to prevent arc eye, a painful condition where the cornea is inflamed. Welding helmets can also prevent retina burns, which can lead to a loss of vision. Both conditions are caused by unprotected exposure to the highly concentrated infrared and ultraviolet rays emitted by the welding arc. Ultraviolet emissions from the welding arc can also damage uncovered skin, causing a sunburn-like condition in a relatively short period of welding. In addition to the radiation, gases or splashes can also be a hazard to the skin and the eyes. Most welding helmets include a window covered with a filter called a lens shade, through which the welder can see to work. The window may be made of tinted glass, tinted plastic, or a variable-density filter made from a pair of polarized lenses. Different lens shades are needed for different welding processes. For example, metal inert gas (MIG) and tungsten inert gas (TIG) welding are low-intensity processes, so a lighter lens shade will be preferred.

While some welding helmets have lens shade built into the helmet (fixed), many welders do not prefer these type because the helmet must be removed each time the user wants to observe the welding area without the darkened lens of the shield. Repeated removals and replacements of the helmet is more time-consuming and can be an annoyance, and can also be dangerous if the welder eschews observation because the welder is tired of removing the entire helmet every time an inspection is required.

The alternative to the fixed lens shade is the pivoting lens shade. A pivoting lens shade can be rotated up so that the welder can observe the weld area, and then flipped back down to continue welding. This process is faster and more convenience than continuously removing the entire helmet and then donning the helmet again. However, one issue that is prominent with the pivoting lens shade is that welding is predominantly a two handed process and utilizing one hand to manipulate the lens shade interrupts the work flow. Additionally, a common practice among welders is to flip the lens shade up and down with an exaggerated neck jerk motion that often results in injury to the neck. Some versions of welding helmets have both a fixed eye shield that may be transparent and a second, pivoting eye shield that is darkened and protects the eye from the bright light of the welding equipment.

U.S. Patent Publication No. 2017/0173720 teaches a welding helmet with a fixed lens shade and a voice actuated heads up display for projecting an image inside the helmet of the welding area. U.S. Pat. No. 7,926,118 discloses another welding helmet with a fixed lens shade and voice actuation for controlling an electric lens to adjust the visibility of the helmet. U.S. Pat. No. 10,913,125 discloses a welding helmet with a fixed lens shade with a face mounted display that receives input from a spatial tracker. U.S. Patent Publication No. 2010/0088793 discloses a welding helmet with a fixed lens shade with a camera and a processor for creating a virtual image that can be used by the welder. U.S. Pat. No. 4,641,292 teaches a welding system that can be voice actuated to control the welding instrument itself. These references provide background to the present invention and their disclosures are incorporated herein by reference in their entireties.

While the foregoing references illustrate the difficulties with welding in a loud, bright, intense environment, none of them teach or suggest the features of the present invention or address the problems of manipulating a pivoting shield in a straightforward manner without interrupting work flow. These and other shortcomings of the existing welding helmets are overcome by the present invention.

### SUMMARY OF THE INVENTION

The present invention is a welding helmet that includes a voice actuated welding screen that is coupled to an actuator for pivoting the screen up (away from the user's field of vision) and down (in place for welding activities) automatically when the proper voice command is given. This handsfree helmet is especially useful is situates where the user is confined to a tight environment, such as beneath a car, etc., where it may be difficult or impossible to manually remove the screen. In a preferred embodiment, the voice actuation can control other features, such as a head lamp to illuminate the welding area by giving a voice command. These and other features of the invention will best be understood with reference the accompanying drawings listed below in conjunction with the inventors' detailed description of the preferred embodiments.

### BRIEF DESCRIPTION OF THE DRAWINGS

FIG. 1 is the elevated, perspective view of a first embodiment of the present invention;

FIG. 2 is an elevated, perspective view of the embodiment of FIG. 1 with the shield up;

FIG. 3 is a front sectioned view of a first embodiment of the present invention; and

FIG. 4 is a schematic view of the elements of the embodiment of FIG. 1.

### DETAILED DESCRIPTION OF THE PREFERRED EMBODIMENTS

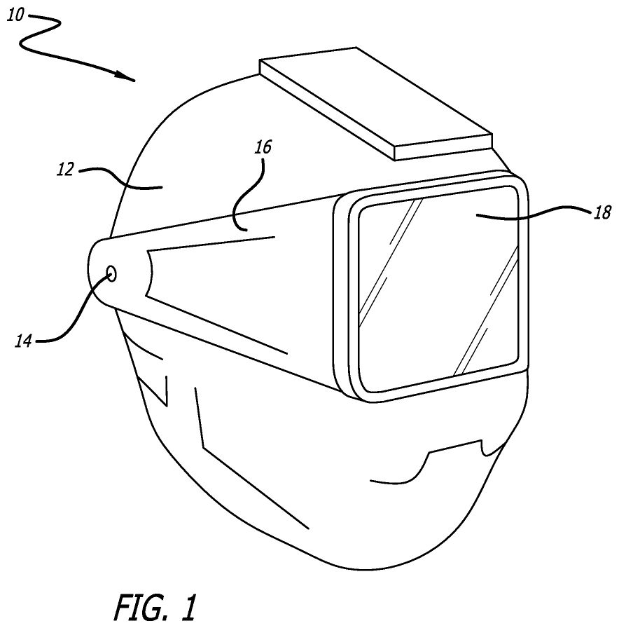

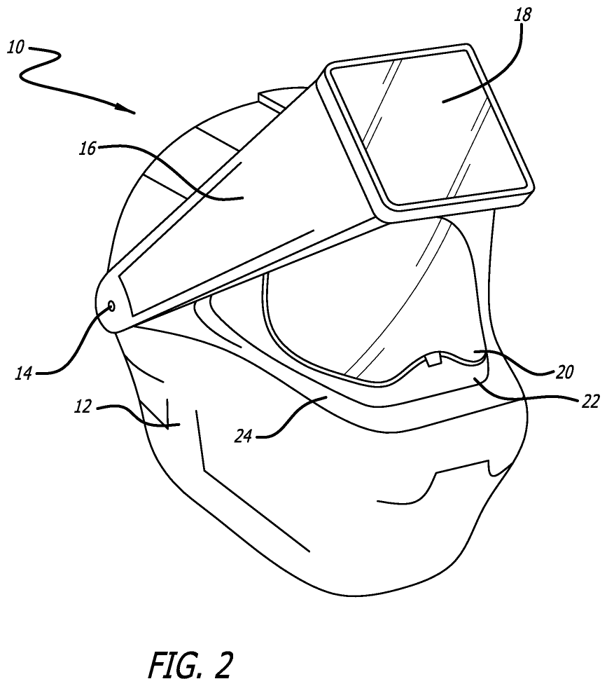

Referring to FIGS. 1 and 2, a first preferred embodiment of the present invention is shown. The helmet 10 includes a hard protective shell 12 and first and second projections 14 extending from each side. On the projections is a reciprocating shield 16 that pivots up and down supported by the projections so as to move in front of (FIG. 1) and above (FIG. 2) a user's line of sight. The shield 16 includes a darkened lens 18 that can be moved into the user's view to protect the user from harmful light during a welding process. Behind the shield 16 is a window 20 (FIG. 2) that allows the user a view of the welding area without the darkened lens while maintaining a protective barrier for the user. The window 20 is inset in a liner 22 build into the shell 12 so as to maintain the integrity of the window/helmet structure. As can be seen in FIG. 2, the shell includes a recessed portion 24 that serves as a landing area for the pivoting shield 16 so that the juncture of the shield 16 and recessed portion forms a smooth and uninterrupted engagement to prevent both light and embers from entering the window area.

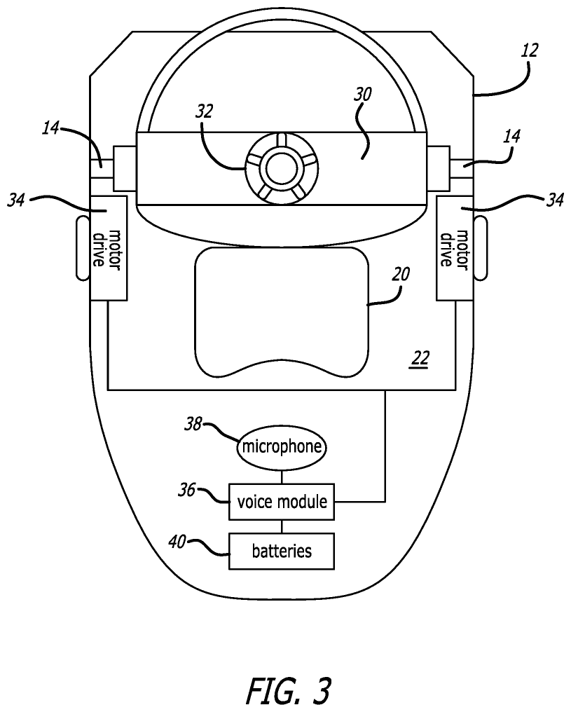

FIG. 3 illustrates the interior components of the helmet 10. An adjustment headband 30 is used to secure the helmet and can be tightened to adjust the fit of the helmet, and it may include a lighting element 32 that projects out of the window to illuminate the working area. On each side of the helmet is a servo motor, solenoid, or electric motor 34 that engage respective sides of the shield 16 to rotate the shield up and down. The motors 34 are controlled by a processor 36 including a voice module 36 that translates audio signals from a microphone 38 and powered by a power supply 40. The processor 36 can receive voice commands from the user, such as “raise” and “lower” and actuate the motors 34 to rotate the shield 16 up and down. This allows a hands free operation of the shield that is not found in any other helmet of this type. The processor can also turn on and off the light 32 using voice commands to allow the user to illuminate the work field without manually turning the light on and off. This is especially practical when wearing heavy gloves or other protective gear that may need to be removed to turn the lights on and off.

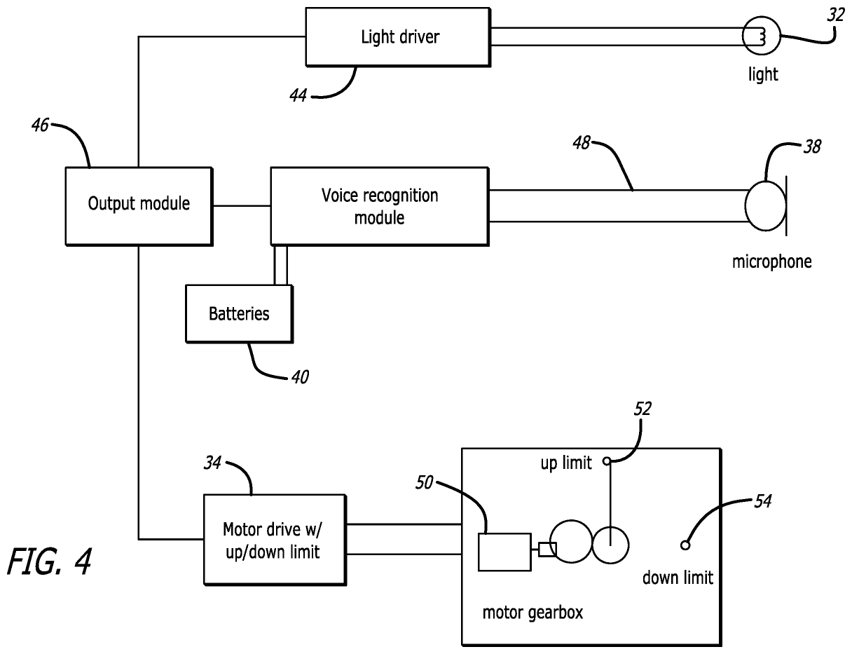

FIG. 4 is a schematic diagram of the elements of the voice actuation aspect of the invention. The light 32 is controlled by a light driver 44 driven by an output module 46. The microphone 38 receives voice commands from the user and the signals are sent along a bus 48 to the processor 36 having a voice recognition module. The processor 36 includes a power supply 40 and converts the voice commands from the voice recognition module to output signals delivered to the output module. The output module delivers a signal to the motor 34 to engage and pivot the shield 16 up or down. The range of arc of the shield is controlled by the gearbox 50 between an upper limit 52 and a lower limit 54 to establish the proper position of the shield 16.

While the inventors preferred embodiments are disclosed and depicted herein, the invention is not limited by these embodiments or the embodiments of the drawings. A person of ordinary skill in the art will readily recognize and appreciate that there are various modifications and substitutions available to the foregoing described embodiments and the present invention includes all such modifications and substitutions. Accordingly, the scope of the invention is properly guided by the construction of the appended claims using their plain and ordinary meanings of the words used.

We claim:

1. A voice actuated personal protective device, comprising:
a helmet configured to be worn on a user's head and including a frontal window;
a reciprocating shield configured to cover the window in a first position and retract over the window in a second position to visually clear the window;
a microphone;
a processor including a voice recognition module for converting audio signals from the microphone to command signals;
an actuator in communication with the voice recognition module, the actuator configured to pivot the reciprocating shield between the first position and the second position based in response to command signals from the voice recognition module; and
a power supply.

2. The voice actuated personal protective device of claim 1, further comprising an illumination device.

3. The voice actuated personal protective device of claim 2, wherein the voice recognition module sends command signals to the illumination device to turn on and turn off according to the audio signals.

4. The voice actuated personal protective device of claim 1, further comprising an adjustment element inside the helmet for adjusting a fit of the helmet.

5. The voice actuated personal protective device of claim 1, wherein the actuator comprises first and second motors adjacent respective pivot points of the reciprocating shield.

6. The voice actuated personal protective device of claim 1, wherein the frontal window includes a transparent face panel.

7. The voice actuated personal protective device of claim 1, wherein the power supply is located inside the helmet.

8. The voice actuated personal protective device of claim 1, wherein the power supply is a battery pack.
